(19) United States
(12) Patent Application Publication
(10) Pub. No.: US 2008/0114549 A1
(43) Pub. Date: May 15, 2008

(54) Rapid response blood analyzer

(76) Inventors: Mark Evan Schafer, Lower Gwynedd, PA (US); Michael Dugery, Doylestown, PA (US)

Correspondence Address: FAY KAPLUN & MARCIN, LLP, 150 BROADWAY, SUITE 702, NEW YORK, NY, 10038, US

(21) Appl. No.: 11/595,409

(22) Filed: Nov. 9, 2006

(51) Int. Cl. G01N 33/48; G01F 25/00; G01R 35/00

(52) U.S. Cl. 702/19; 702/104; 702/106

(57) ABSTRACT

A system for estimating clotting properties of blood, comprises a sensor generating an initial data signal associated with a measurement of clotting of the blood during an initial time period prior to a final clotting time of the blood and a processor determining parameters of an equation to curve fit the initial data signal, extrapolating the equation forward in time to a time at which a desired level of clotting is achieved and estimating from the extrapolated equation the final clotting time of the blood.

### BACKGROUND

There are several causes of deep vein thrombosis, or the formation of blood clots that travel through the blood stream. If such a clot becomes lodged in an artery, the supply of blood therethrough may be reduced or cut off entirely resulting in a stroke, pulmonary embolism or other serious condition brought on by the cessation of blood flow to portions of the body downstream of the clot.

Patients at high risk for thrombotic disease are often treated with anticoagulants, such as antithrombins and antiplatelet agents. To ensure application of the appropriate dosage of the medication, the hemostatic status of these patients is assessed at multiple intervals by, for example, measuring the clotting speed of a blood sample. However, current systems for monitoring the hemostatic status of these patients are often complex and/or time consuming making long term monitoring inconvenient and expensive.

### SUMMARY OF THE INVENTION

In one aspect the present invention is directed to a system for estimating clotting properties of blood, comprising a sensor generating an initial data signal associated with a measurement of clotting of the blood during an initial time period prior to a final clotting time of the blood and a processor determining parameters of an equation to curve fit the initial data signal, extrapolating the equation forward in time to a time at which a desired level of clotting is achieved and estimating from the extrapolated equation the final clotting time of the blood.

### BRIEF DESCRIPTION OF DRAWINGS

FIG. 1 is a graph showing a short time coagulation analysis of samples with different levels of heparin;

FIG. 2 is a graph showing plasma test results compared to a curve fit of the data;

FIG. 3 is a diagram showing the displacement due to vibration through a crystal and the adjacent fluid;

FIG. 4 is a perspective view showing an exemplary quartz resonator plate sensor according to the invention;

FIG. 5 is a top view showing a direction of oscillation of the sensor shown in FIG. 4;

FIG. 6 is a top view showing a resonant pattern of activity at the surface of the sensor shown in FIG. 4;

FIG. 7 is a diagram showing an amplitude distribution of the resonant pattern for the sensor shown in FIG. 4;

FIG. 8 is a top view showing a sensor having a pattern of process accelerant in a first orientation;

FIG. 9 is a top view showing a sensor having a pattern of process accelerant in a second orientation;

FIG. 10 is a graph showing signal level vs. sensor coverage for an exemplary sensor;

FIG. 11 is a perspective view showing another embodiment of a sensor having two resonator plates;

FIG. 12 is a side view of the sensor shown in FIG. 11;

FIG. 13 is a diagram showing multiple side views of blood traveling through the sensor shown in FIG. 11; and

FIG. 14 is a top view showing a different embodiment of a single quartz element sensor having multiple sensor regions.

### DETAILED DESCRIPTION

The present invention may be further understood with reference to the following description and to the appended drawings, wherein like elements are referred to with the same reference numerals. The present invention relates to devices for measuring parameters of a fluid and, in particular, devices which measure coagulation parameters of blood.

Exemplary embodiments of the present invention provide a system and method for rapidly and conveniently determining clotting properties of a blood sample which is operable without extensive training. For example, in one embodiment an estimate of the clotting properties of the blood is obtained in about 30 seconds—before the sample has reached its final clotted condition. In contrast, tests that require a user to wait until the blood has completely clotted may require significantly longer periods of time to complete, especially when the blood has been treated with anticoagulant agents such as heparin, even where the testing process involves the addition of clotting accelerants.

According to the embodiments of the present invention, the eventual clotting time of a sample of blood is projected based on data derived from the initial stages of clotting. Changes over time in a signal corresponding to, for example, vibrational characteristics of the blood, are analyzed to determine a final clotting time of the sample. For example, the signal may be derived from vibrational characteristics of an acoustic piezoelectric quartz sensor in contact with the sample of blood. However, those skilled in the art will understand that other types of sensors generating appropriate signals associated with a measure of clotting of the blood may be used instead of an acoustic sensor.

The initial stages of the clotting process of blood may be represented using a sigmoid-shaped response curve from the quartz sensor. The form of the sigmoid equation S(t) is described by the following function:

S(t)=1/(1+e^{-a(t-b)})

### In the equation t represents time, and a, b are factors that shift and modify the curve to fit the data generated by the sensor.

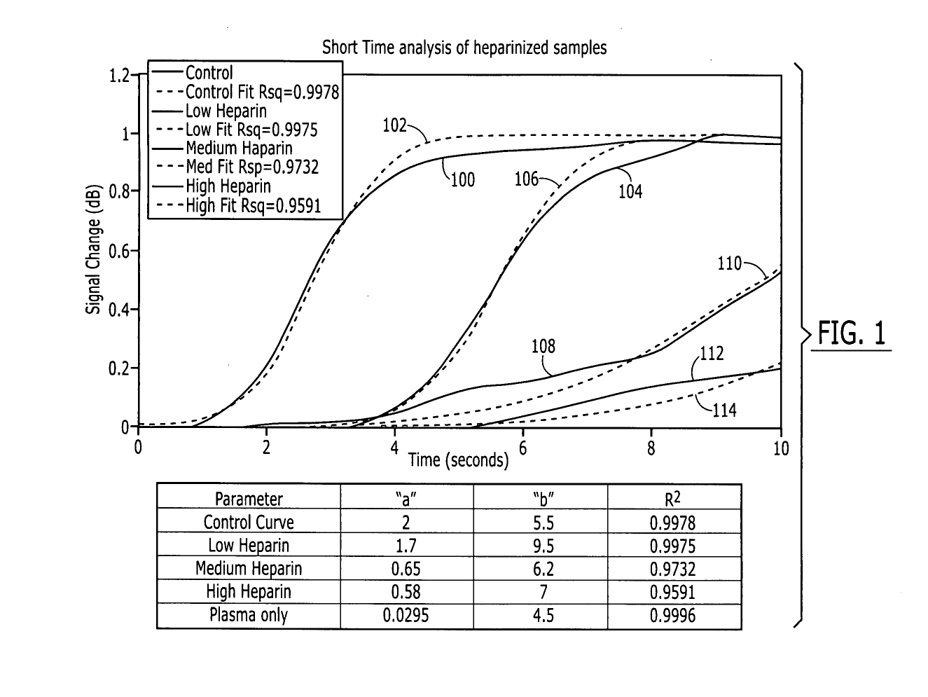

FIG. 1 shows data from an experiment using whole pig blood having various amounts of heparin added to simulate the administration of anti clotting agents. In the test, thromboplastin was used as a clotting accelerant, so that the clotting began in less than 30 seconds. However, the purpose of the curve fitting is to further reduce the time required to estimate a dosage of heparin required to obtain desired clotting properties. Thus, according to this embodiment of the invention, properties of the blood during the first 10 seconds after the clotting accelerant has been mixed in are analyzed. Measured data is shown by the solid lines in FIG. 1 which represent a control group of blood with no heparin 100, a low level 104, a medium level 108 and a high level 112 of heparin in the blood.

The dashed lines 102, 106, 110 and 114 shown in FIG. 1 represent the sigmoid functions used to curve fit the experimental data described above. The two parameters a and b are adjusted to fit the data. The coefficient of determination (R² value) is also indicated for the curves. This parameter shows how well the data and the curve fit function match. The closer the value of R² is to 1.0, the better the match.

The table of values below the graph in FIG. 1 gives the specific values of a and b used to generate the corresponding sigmoid curves of the curve fit. Along those values, the correlation parameter R² is also displayed. These parameters can be analyzed to further predict the relative degree of heparin in the blood being tested. Although the estimate may be accurately conducted in less than 10 seconds, some experimental noise was present in the curves, which reduced the accuracy of the fit.

The precise values of a and b, which are determined experimentally for each sensor configuration used, depend in part upon the concentration and type of accelerant used, the size of the sensors, the geometry of the setup and other parameters. Once the values of a and b have been determined for a sensor configuration, the curve fit may be used to rapidly estimate the heparin concentration in subsequent blood samples. According to the invention there is no need to wait until the blood actually forms a complete clot to determine a desired dosage of heparin or other blood thinner medication and, consequently, to determine if a current dosage level needs to be adjusted.

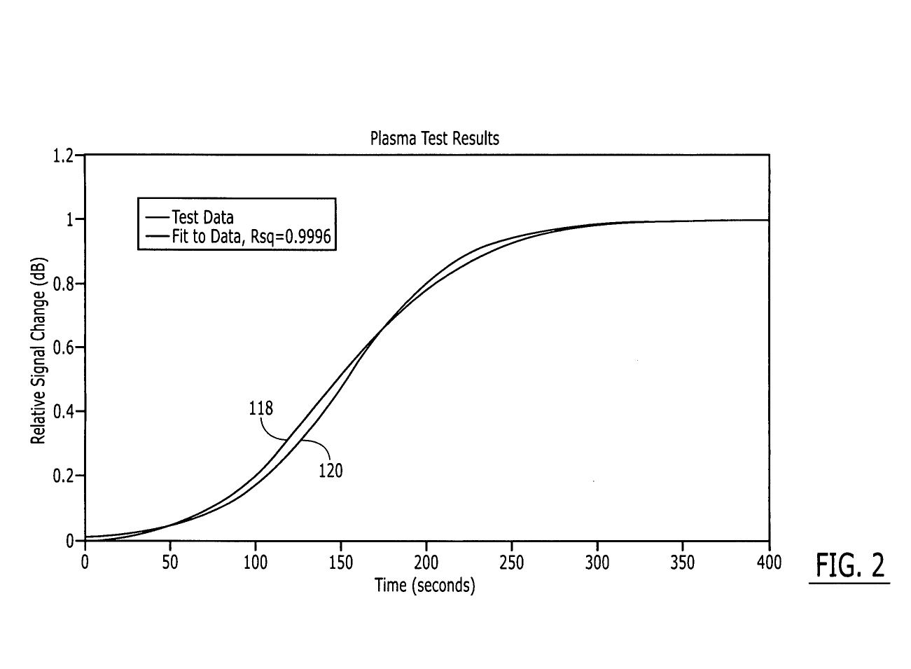

A second experiment was performed to further validate the application of sigmoid function curve fitting according to the present invention to rapidly predict the coagulation rate of blood. Recalcified plasma was placed in a small sample volume chamber without the addition of an accelerant. The natural clotting time in this experiment was approximately 65 times longer than the clotting time in the experiment described above. FIG. 2 shows the test data represented by line 118 for the clotting time, and the sigmoid curve fit represented by line 120. As shown by the “Plasma Only” entry in the table of FIG. 1, the value of a for the second experiment is approximately 65 times lower than the value of a for the control case with whole blood.

According to the embodiments of the present invention, the change in the measured parameter of the blood is continually analyzed to determine the matching sigmoid function parameters a and b to compute the curve fit. When the predicted values from the curve fit and the measured values of the parameter of interest fall within a desired tolerance for a preset period of time, the determination of the parameters a and b may be halted. The resulting sigmoid function may be used to estimate the final clotting time of a blood sample under conditions similar to those for the test. The time necessary to carry out the estimation may vary with the condition of the blood. For example, blood which clots more slowly will result in a longer estimation time. However, the time required to determine clotting time using conventional systems is also extended under these conditions. Thus, for all conditions the exemplary procedure reduces the time required to produce an accurate estimate of the clotting time.

As indicated above, the sigmoid function curve fit may be used to estimate the clotting time based on a signal generated by a sensor interrogating a blood sample. In one exemplary embodiment, the sensor is an acoustic quartz sensor operating in a thickness shear mode to produce a pattern of lateral vibration on the surface of the sensor. In addition, in some embodiments a bioactive accelerant (e.g., thromboplastin) which may be disposed as a surface treatment on the sensor is used to speed up coagulation.

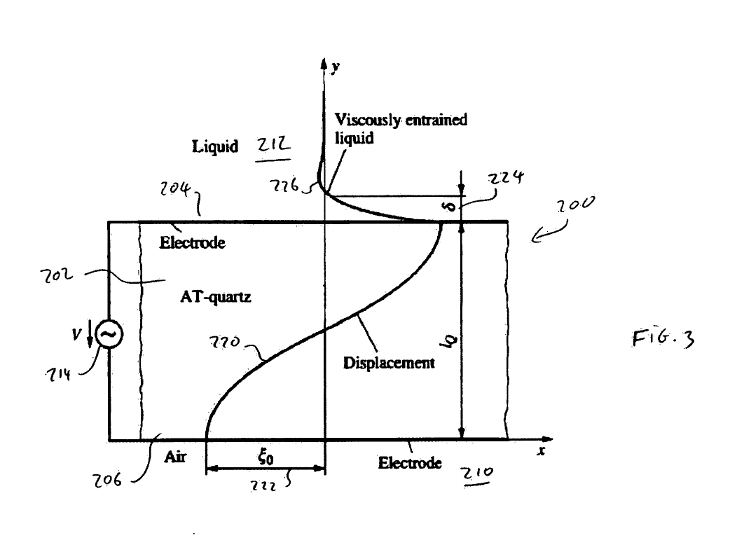

According to the invention, the exemplary sensor operates by the interaction of the vibration of the sensor surface with the fluid (blood in this case) in contact therewith. Vibrations in the quartz crystal are directed according to the orientation of its crystalline structure and therefore may be tailored to select a desired vibration orientation. FIG. 3 shows a diagram of an exemplary sensor 200 placed between air 210 and a fluid 212 the properties of which are to be measured. The sensor 200 comprises a circular quartz crystal 202 between two electrode regions 204, 206 having a voltage applied thereto by a power source 214.

The line 220 represents displacement due to the vibration of the crystal driven by the voltage, having a maximum amplitude 222 at the surface of the electrodes 204, 206. The line 226 represents the displacement due to the vibration transmitted to the fluid 212 from the sensor 200. The amplitude of the displacement in the fluid 212 decays rapidly as one moves away from the surface of the sensor 200, and is near zero outside of the thickness 224 of the boundary layer. The vibration of the crystal may be converted to a data signal using a conventional network analyzer (e.g., Agilent Corp. Model 4195) or similar device. In another embodiment, the crystal may be electrically incorporated into a resonant oscillator circuit (also known as a resonant crystal circuit) with the frequency and damping factor of the oscillator indicating the vibration characteristics of the crystal.

FIG. 4 shows an exemplary layout of a quartz crystal sensor with two electrodes according to the present invention. The sensor 250 comprises a substantially circular disk-like crystal 252 (for example a quartz crystal) with top and bottom surfaces that are substantially planar. A top electrode 254 and a bottom electrode 256 are disposed on opposing surfaces. Each electrode comprises an electrical contact 258 connected to a power source (not shown). One or both of the planar surfaces may be exposed to the blood that is to be tested.

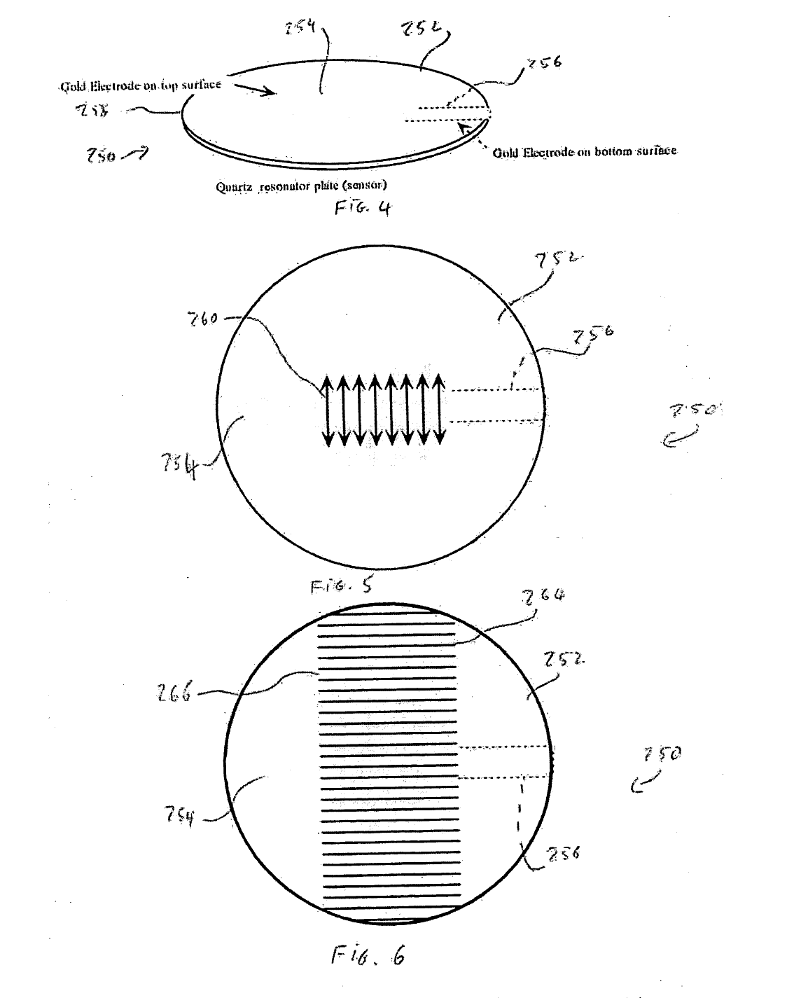

As indicated in FIG. 5, lateral vibrations within the crystal 252 occur in only one direction along the surface of the crystal 252 as indicated by the arrows 260. As indicated above, the direction of vibration is a function of the structure of the crystal 252 with a resonant pattern of activity on the surface of the crystal 252 as shown schematically in FIG. 6. The lines 264 represent areas of high vibration on the surface, while the spaces 266 between lines 264 represent areas of little or no vibration.

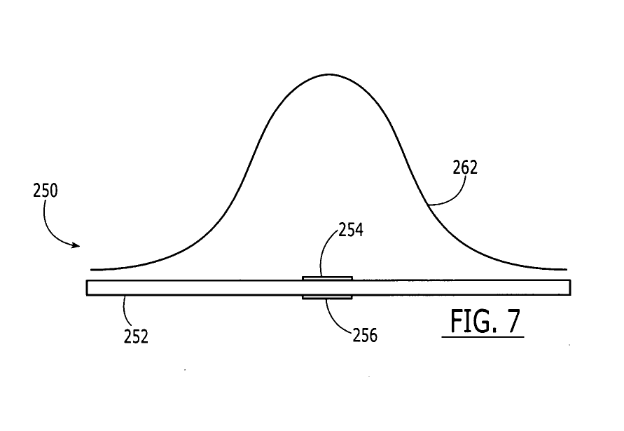

The distribution of amplitude for the vibration induced in the crystal 252 is shown schematically in FIG. 7. The curve 262 represents the vibration amplitude seen along a slice of the sensor 250 taken along a diameter thereof. Because the sensor 250 is substantially circular, the curve 262 is substantially symmetrical about the center of the sensor 252 with a maximum amplitude over the electrodes 254, 256 as that area is actively driven electrically. The amplitude is lower in peripheral regions without electrodes, more remote from the applied electrical driving force. It should be noted that the vertical height of the curve 262 above the sensor 250 is a graphical representation of the lateral vibration amplitude that exists at that point on the crystal surface.

The precise position and magnitude of the resonant vibration pattern illustrated by the curve 262 is a function of the electrode 254, 256 geometry, the geometry of the crystal 252, the orientation of the crystalline structure of the crystal 252, and any imperfections in the components, etc. Thus, many of the parameters shaping the resonant vibration pattern are not easily controlled. In one embodiment of the invention, the relative change in resonant characteristics caused by the loading of the fluid on the surface of the sensor 250 is measured. However, when relative changes in the output of the sensor are considered, exact details of the vibration patterns are of less concern and it is not necessary to build the sensor to extreme tolerances.

In another exemplary embodiment of a sensor according to the invention, it is desirable that a process accelerant material be placed on a surface of the sensor. As discussed above, the accelerant may be thromboplastin which speeds coagulation, reducing the time required to determine coagulation properties. The accelerant may be placed on the sensor as a continuous coating or as a pattern on portions of the surface of the sensor. Utilizing a pattern rather than a continuous coating of accelerant may be advantageous, because less accelerant is necessary, and a greater surface area of accelerant per unit volume of accelerant is available to interact with the fluid of interest.

When the coating is applied in a non-continuous manner, the pattern is selected to minimize negative interaction with surface vibration patterns of the sensor. For example, if the pattern is formed as strips of accelerant material interposed with strips of bare sensor surface, the orientation of the strips is of great importance.

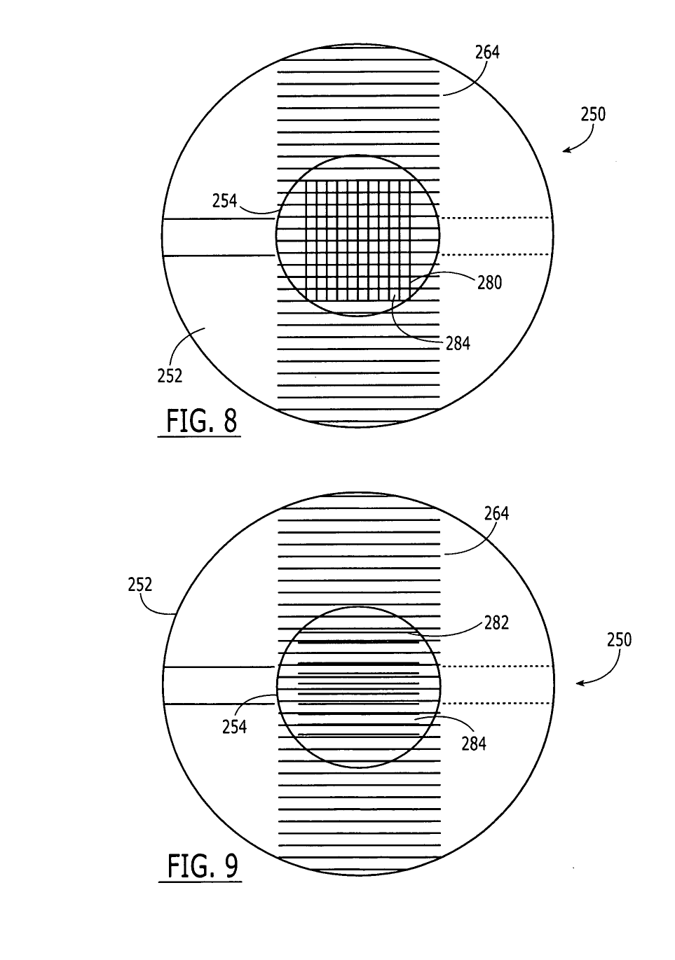

As shown in FIG. 8, strips 280 of accelerant material together with the portions of bare sensor 284 are oriented substantially perpendicular to an expected direction of vibration, shown schematically by lines 264 reducing the potential for negative interference of the strips 280 with the resonant pattern of the sensor 250 as the orientation of the strips substantially perpendicular to the resonant vibration pattern make the loading of the accelerant non-periodic with respect to the resonant vibration pattern.

Alternatively, the strips 282 of accelerant material may be placed substantially parallel to a direction of vibration, as in the embodiment shown in FIG. 9. In this configuration, the loading of the accelerant imposes a second periodic structure on the resonant vibration pattern. However, interaction of the vibrations due to the accelerant strips and to the resonant vibration pattern of crystals is difficult to predict and control. Thus, it is difficult to manufacture sensors of this configuration with consistent properties, without large manufacturing variability.

Those of skill in the art will understand that the pattern and location of the accelerant may be varied without departing from the scope of the invention. For example, the accelerant material may be placed on the surface of the sensor as individual spots rather than strips. The pattern of accelerant may have other shapes as well, however care should be taken in evaluating the relationship between the accelerant pattern and the resonant vibration pattern of the crystal. In general, a preferred pattern is non-periodic in the direction of vibration of the crystal.

When incorporating the sensors according to the invention in to a commercial blood testing apparatus, it may be desirable to provide a system for calibration of the sensor. A self-calibration system for the blood coagulation time measurement may be used to correct for imprecisions in the manufacturing of the sensor, thus reducing the time and cost of manufacture. In addition, the self-calibration system may be used to compensate for the small magnitude of the measured signal compared to background signals received by the sensor.

When blood properties are measured by the sensor, the effect of the blood flowing over the sensor, or loading of the liquid blood, is typically in the order of about 14 to 18 dB. This refers to the change in amplitude of the vibration of the sensor crystal, or in the resistance of the resonant crystal circuit. The change is brought about by the passage of blood over the surface of the sensor, compared to the response of the sensor without any blood flow over it. In contrast, the change in signal brought about by the coagulation of the blood on the sensor is in the order of about 0.9 to 1.4 dB. There is thus about a 500% difference between the magnitude of the signal of interest due to clotting and that of the change in the signal caused solely by the contact between the blood and the sensor.

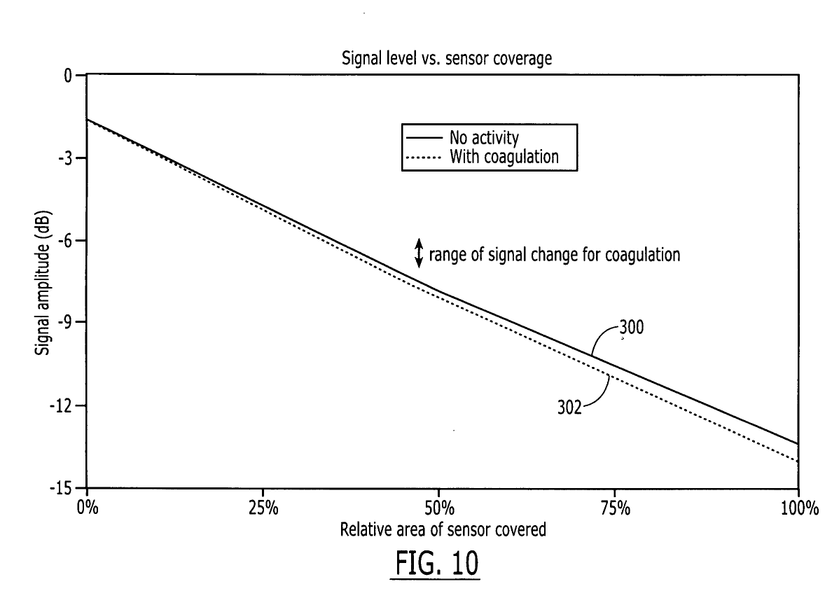

FIG. 10 illustrates the problem of the relatively small magnitude of the measurand signal. As the blood (or other fluid) progressively covers the surface of the sensor, the signal amplitude changes significantly. In the exemplary case shown, the change is about 12 dB as the blood covers from 0% to 100% of the sensor, shown by the solid line 300. The dashed line 302 represents the signal change when the blood is mixed with a coagulation accelerant just before being applied to the surface of the sensor. In this case the blood progressively covers the sensor as it is undergoing coagulation. Because the difference between the signals shown by lines 300, 302 is so small, it is difficult to distinguish between the signal change due to coverage by the blood and that due to the change in properties of the blood.

The sensor according to embodiments of the present invention is self-calibrating in the sense that the signal of interest is derived form the total signal of the sensor by removing an interfering load signal due to blood contacting the sensor. For example, a dual sensor arrangement may produce a difference signal usable to accentuate the desired measurand signal. More specifically, one embodiment would have one sensor coated with a clotting accelerant as described above, while the other has no accelerant coating. In one embodiment, the dual sensor arrangement is formed by two separate quartz crystal sensors. Alternatively, the two sensors may be formed from a single quartz crystal with appropriate electrode patterns deposited thereon.

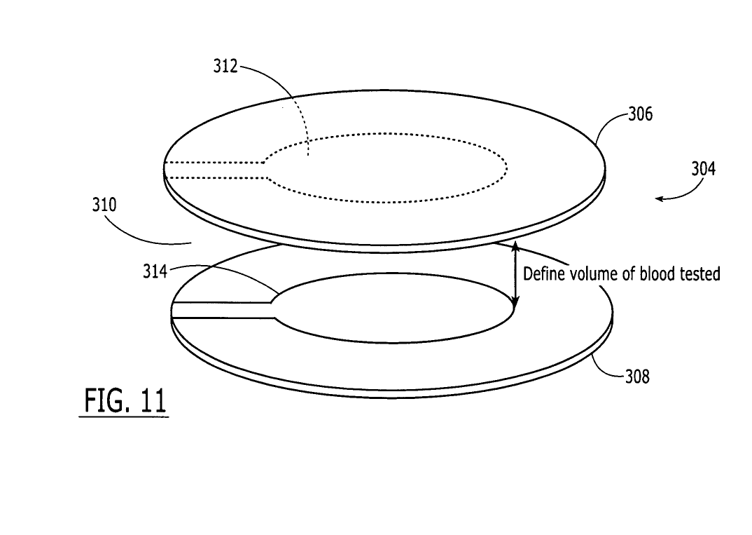

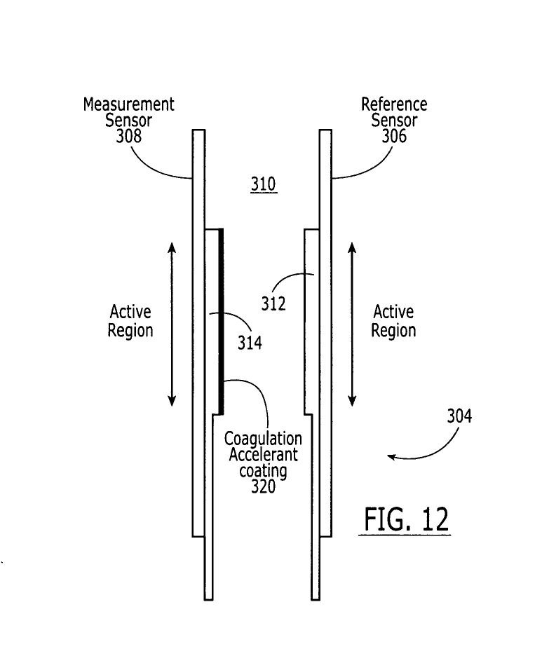

An exemplary embodiment of the dual sensor device according to the invention is shown in FIGS. 11 and 12. The sensor 304 is configured to detect the signal due to blood clotting that is several orders of magnitude smaller than the effect of the fluid loading. The quartz resonator sensor utilized in the embodiment is designed to resonate in an unloaded condition with no fluid on its surface. The resonance is very sensitive to load changes, thus making the device very precise in measuring small signals. However, when small signals are masked by larger signals, the device becomes significantly less sensitive if only a single sensor is used.

According to the invention, multiple sensors are used with, in this embodiment, a reference sensor 306 mounted substantially parallel to a measurement sensor 308. A passage 310 is formed between opposing surfaces of the sensors 306, 308 to allow passage of the blood or other fluid and a reference electrode 312 and a measuring electrode 314 are mounted on the opposing surfaces of, respectively, the reference sensor 306 and the measuring sensor 308. The measurement sensor 308 also comprises a coagulation accelerant material 320 such as, for example, a coating of thromboplastin, disposed in a pattern such as parallel stripes. In this embodiment both sensors are exposed to the blood, but only one sensor has the coagulation accelerator on its surface.

By considering only the difference in output between the two sensors 306, 308 instead of the absolute magnitude of the sensor signal, the desired measurand signal is determined. The larger signal change due to the blood load (over 20 dB) is cancelled out, emphasizing the much smaller signal change due to the thrombin activation (about 1.5 dB). In this embodiment it is possible to measure signal changes due to coagulation without resorting to more expensive highly calibrated sensors. For example, an electronic processor may be used to manipulate signals received from the two sensors to derive a measurand signal substantially uninfluenced by the interference load signal.

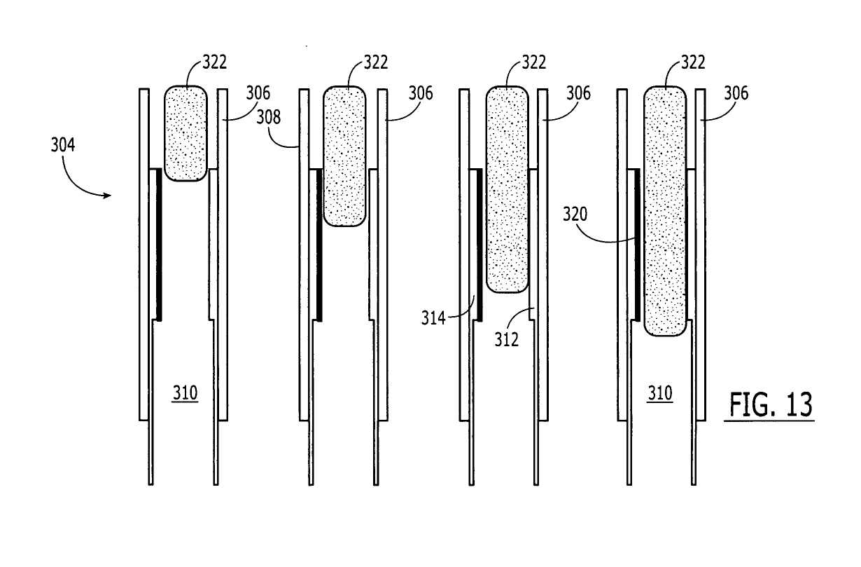

FIG. 13 shows the movement of blood through a dual sensor blood properties measuring device according to an exemplary embodiment of the invention. Blood 322 is introduced into the test chamber 310 formed between opposing surfaces 306, 308 of the sensor 304. As described above, only the measuring sensor 308 has a coating 320 which, in this embodiment, comprises a bio-reaction accelerant. The difference in the signal detected by the two sensors 306, 308 is used to differentiate the portions of the signal corresponding to the bioreaction, in this case the coagulation of the blood.

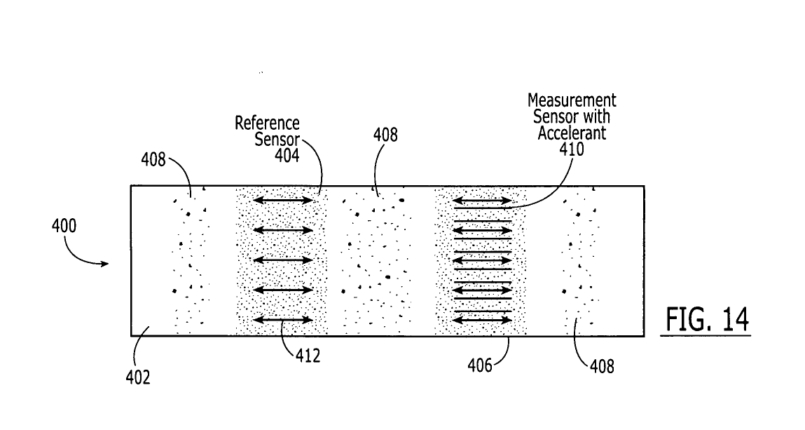

To reduce the additional cost associated with the dual sensor design, in a different embodiment, multiple sensor regions are formed on a single quartz sensor. For example, as shown in FIG. 14, sensor regions are defined by appropriate electrode patterns placed on the crystal plate. The exemplary sensor 400 comprises a single quartz crystal plate 402 divided into a reference sensor region 404 and a measurement sensor region 406 by placing the appropriate electrodes on those regions. Ground regions 408 are located therebetween to control surface vibration patterns as would be understood by those skilled in the art. In addition, the measurement sensor region 406 may comprise an accelerant coating 410, as described above.

The direction of the surface movement of the two sensor regions is depicted by the arrows 412. In this exemplary embodiment, the vibration patterns are aligned such that there is some cross talk between the sensor regions 404, 406. As would be understood by those skilled in the art, the cross talk may be reduced or promoted, depending on the design requirements of the measuring device.

To isolate the vibration patterns of the two sensor regions 404, 406 and reduce their interaction, either one of the quartz plates of the sensors may be oriented to vibrate at 90 degrees from the orientation shown in FIG. 14—i.e., so that a direction of vibration is substantially parallel to an axis along which the regions 404, 406 are separated from one another. This may be beneficial, for example, in long term measuring sessions. Alternatively, the sensor regions 404, 406 may be interrogated in an alternating manner, such that they are never simultaneously active. For example, the sensors 404, 406 may be interrogated sequentially.

in another embodiment, it may be desirable to have cross talk between the sensors. For example, one of the sensors may be a passive receiver while the other transducer is energized. The received signal may thus be used as a calibration indicator showing, for example, the progress of the blood over the sensor 400 between the two active regions 404, 406.

The present invention has been described with reference to specific exemplary embodiments. Those skilled in the art will understand that changes may be made in details, particularly in matters of shape, size, material and arrangement of parts. Accordingly, various modifications and changes may be made to the embodiments. For example, the exemplary devices described may be used to evaluate different properties of various fluids. The specifications and drawings are, therefore, to be regarded in an illustrative rather than a restrictive sense.

What is claimed is:

1. A system for estimating clotting properties of blood, comprising:
a sensor generating an initial data signal associated with a measurement of clotting of the blood during an initial time period prior to a final clotting time of the blood; and
a processor determining parameters of an equation to curve fit the initial data signal, extrapolating the equation forward in time to a time at which a desired level of clotting is achieved and estimating from the extrapolated equation the final clotting time of the blood.

2. The system according to claim 1, wherein the equation is a sigmoid equation of the form S(t)=1/(1+e^{-a(t-b)}), wherein t represents time and a, b are the parameters.

3. The system according to claim 2, wherein the parameters a and b are determined based on the initial data signal.

4. The system according to claim 1, wherein the sensor is an acoustic crystal sensor.

5. The system according to claim 1, wherein the sensor comprises two acoustic sensors.

6. The system according to claim 1, wherein initial parameters are determined based on a selected sensor configuration.

7. The system according to claim 2, wherein the parameters a and b are varied to curve fit the sigmoid function to the initial data signal within a selected coefficient of determination.

8. A device for measuring properties of a fluid, comprising:
a sensor element having a fluid contacting surface, the sensor element including a first crystal;
electrodes disposed on the crystal to induce a resonant vibration pattern; and
a signal generator generating signals corresponding to the vibration of the crystal.

9. The device according to claim 8, wherein the sensor element comprises a substantially planar quartz crystal.

10. The device according to claim 9, wherein the first crystal is substantially circular.

11. The device according to claim 8, further comprising a biological accelerant disposed in a pattern on the sensor element.

12. The device according to claim 11, wherein the biological accelerant is disposed on the sensor element in a pattern of substantially parallel strips.

13. The device according to claim 11, wherein the biological accelerant is disposed on the sensor element in a pattern of discrete dots.

14. The device according to claim 11, wherein the biological accelerant is disposed in a pattern that is non periodic relative to the resonant vibration pattern.

15. The device according to claim 12, wherein the substantially parallel strips are substantially perpendicular to the resonant vibration pattern.

16. The device according to claim 11, wherein the biological accelerant comprises thromboplastin.

17. The device according to claim 8, wherein the electrodes are disposed on opposing surfaces of the first crystal to define an electrically driven resonant region.

18. The device according to claim 8, wherein the signal generator produces a data signal corresponding to coagulation properties of blood.

19. The device according to claim 17, wherein the data signal is generated before a final clotting time of the blood.

20. The device according to claim 8, further comprising a second sensor element cooperating with the sensor element to measure coagulation properties of blood.

21. The device according to claim 8, further comprising a second sensor element to provide error correction of the data signal.

22. The device according to claim 20, wherein the second sensor element comprises a second crystal.

23. The device according to claim 20, wherein the second sensor element comprises a separate region of the first crystal.

24. A self calibrating blood coagulation measuring device, comprising:
a measurement sensor having a measurement electrode mounted thereon, the measurement sensor generating a data signal containing a measurand signal and an interfering load signal;
a reference sensor having a reference electrode mounted thereon, the reference sensor generating a data signal containing the interfering load signal;
a flow chamber formed between the measurement sensor and the reference sensor, wherein the blood simultaneously covers at least portions of the measurement sensor and the reference sensor; and
a coagulation accelerant coating disposed on the measurement sensor.

25. The measuring device according to claim 24, further comprising a processor manipulating the data signals from the measurement sensor and the reference sensor to derive the measurand signal by canceling the interfering load signal.

26. The measuring device according to claim 24, wherein the measurement sensor and the reference sensor comprise quartz crystal elements.

27. The measuring device according to claim 24, wherein the measurement sensor and the reference sensor are substantially parallel quartz crystal sensors.

28. The measuring device according to claim 27, wherein the substantially parallel measurement and reference sensors are separated by a distance sufficient to permit flow of blood therebetween.

29. The measuring device according to claim 24, wherein the measurement sensor and the reference sensor comprise a single quartz crystal with a pattern of electrodes coupled thereto defining a measuring region and a reference region on the crystal.

30. The measuring device according to claim 29, wherein a ground region separates the measuring region from the reference region.

31. The measuring device according to claim 24, wherein the reference sensor and the measuring sensor vibrate in substantially identical directions.

32. The measuring device according to claim 24, wherein the reference sensor and the measuring sensor vibrate in directions substantially perpendicular to one another.

33. The measuring device according to claim 24, wherein the measurement sensor and the reference sensor are interrogated in an alternating manner to reduce crosstalk therebetween.

34. The measuring device according to claim 24, wherein one of the measuring and the reference sensors is energized while the other operates as a passive receiver to obtain a calibration signal.

35. The measuring device according to claim 25, further comprising a processor performing a curve fit of a sigmoid equation to an initial data signal from the measurand signal to estimate a final coagulation time.

36. The measuring device according to claim 25, wherein the signal from the measurement sensor is between about 14 dB and 18 dB, and the measurand signal is between about 0.9 dB and 1.4 dB.

37. A method of computing blood properties, comprising:
receiving an initial data signal from a sensor in contact with the blood, the initial data signal being measured before a final clotting time of the blood;
computing parameters a and b of a sigmoid equation of the form S(t)=1/(1+e^{-a(t-b)}) to fit the initial data signal; and
estimating the final clotting time of the blood from the sigmoid equation with computed a and b parameters.

38. The method according to claim 37, further comprising repeatedly calculating the a and b parameters until a coefficient of determination with the initial data signal is within a selected value.

39. The method according to claim 37, further comprising receiving the initial data signal from a quartz crystal sensor having a pair of electrodes, a surface of the quartz crystal being in contact with the blood.

40. The method according to claim 37, further comprising receiving a measurand signal derived from a difference signal accentuating the measurand signal by canceling out an interfering load signal.
